(19) United States
(12) Patent Application Publication
(10) Pub. No.: US 2016/0310110 A1
(43) Pub. Date: Oct. 27, 2016

(54) Acquisition control for mixed mode ultrasound imaging

(71) Applicant: Siemens Medical Solutions USA, Inc., Malvern, PA (US)

(72) Inventors: Stirling Dodd, Monroe, WA (US); Chi Hyung Seo, Sammamish, WA (US); King Yuen Wong, Issaquah, WA (US)

(21) Appl. No.: 14/694,859

(22) Filed: Apr. 23, 2015

(51) Int. Cl. A61B 8/00; A61B 8/14; A61B 8/08

(52) U.S. Cl. CPC A61B 8/54; A61B 8/488; A61B 8/486; A61B 8/14

(57) ABSTRACT

For mixed mode imaging, the ultrasound scanner distinguishes between times when different modes of imaging are appropriate based on motion or transducer usage. Any switching between modes, such as between B-mode and mixed mode imaging, occurs automatically based on the detection by the ultrasound scanner of motion, alleviating the need for sonographer manual selection.

### BACKGROUND

The present embodiments relate to mixed mode ultrasound imaging. In a typical ultrasound scanning session, the sonographer frequently switches between B-mode and mixed mode imaging. Mixed modes may include B-mode in combination with color flow (i.e., Doppler mode). One reason for frequent switching of the modes, especially for low-end systems, is that B mode image quality and/or frame rate is reduced while in a mixed mode due to hardware and/or software limitations. To find a region for mixed mode imaging, the user selects B-mode imaging with the corresponding better frame rate and/or image quality. Once found, the user then selects mixed mode imaging with the lesser B-mode image quality but additional imaging mode. The process may be repeated in an imaging session. This type of workflow greatly increases exam duration.

### BRIEF SUMMARY

By way of introduction, the preferred embodiments described below include methods, instructions, and systems for acquisition control for mixed mode imaging. The ultrasound scanner distinguishes between times when different modes of imaging are appropriate based on motion or transducer usage. Any switching between modes, such as between B-mode and mixed mode imaging, occurs automatically based on the detection by the ultrasound scanner of motion, alleviating the need for sonographer manual selection.

In a first aspect, a method is provided for acquisition control for mixed mode ultrasound imaging. A patient is scanned in B-mode. B-mode images are generated from the scanning in the B-mode. The motion of the ultrasound transducer, the patient being scanned by the ultrasound transducer or both transitioning to below a threshold is detected. The scanning of the patient automatically switches to a mixed mode in response to the detecting that the motion is below the threshold. The mixed mode is different from the B-mode. Mixed mode images are generated from the scanning in the mixed mode.

In a second aspect, a non-transitory computer readable storage medium has stored therein data representing instructions executable by a programmed processor for acquisition control for mixed mode ultrasound imaging. The storage medium includes instructions for distinguishing between a search for a region of interest and imaging the region of interest (the distinguishing being based on input from a transducer motion sensor, ultrasound data, or both, imaging in only B-mode for the search), and imaging in the mixed mode for the imaging of the region of interest.

In a third aspect, a system is provided for acquisition control for mixed ultrasound imaging. A transmit beamformer and a receive beamformer are configured to scan, with a transducer, a patient with ultrasound in B-mode and color flow mode. A processor is configured to cause the transmit and receive beamformers to operate in the B-mode and not the color flow mode during transducer motion and to operate in both the B-mode and color flow mode for stationary positioning of the transducer. A display is operable to display a B-mode image without a color flow image during the transducer motion and to display both the B-mode image and the color flow image for the stationary positioning of the transducer.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for acquisition control for mixed mode ultrasound imaging;

FIG. 2 illustrates an example of transitioning between B-mode scanning and mixed mode scanning; and

FIG. 3 is one embodiment of a system for acquisition control for mixed mode ultrasound imaging.

### DETAILED DESCRIPTION OF THE DRAWINGS AND PRESENTLY PREFERRED EMBODIMENTS

The workflow is improved in mixed mode medical ultrasound imaging. During “search” or “seek” mode, the sonographer tries to find the best angle or transducer window to the region of interest. In this search mode but while in a mixed mode configuration, mixed mode is temporarily disabled despite being configured for mixed mode imaging. A superior B-mode image quality is provided to find the best scan position with a higher frame rate. During “flow” or “scan” mode, the mixed mode is enabled. This removes the need for the operator to switch to B-mode before returning to the mixed mode or vice versa.

In one embodiment, automatic switching between B and color flow modes turns color flow mode on and off based on the user being in search mode or scan mode. The search mode may be detected by, but not limited to, frame correlation, tissue contact, and/or brightness of the image. B-mode quality and/or frame rate are restored during search mode to increase user responsiveness. The automated switching between modes despite being configured for mixed mode may reduce exam time and reduced sonographer fatigue from manual switching.

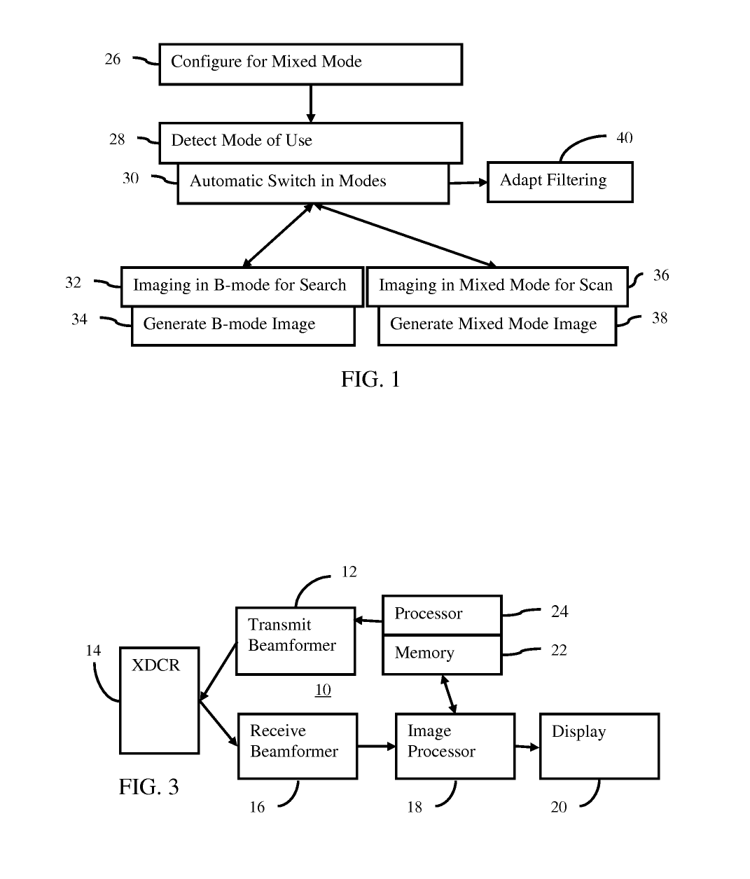

FIG. 1 shows a method for acquisition control for mixed mode ultrasound imaging. The mode of use of the transducer is detected as part of mixed mode imaging. The mode of use of the transducer is different from the mixed mode of imaging. The mixed mode of imaging uses two or more imaging modes. The mode of use of the transducer is how the transducer is used regardless of the imaging mode. The transducer may be used to search for a region of interest and to scan the located region of interest. In response to the transducer being used to search, the mixed mode is suspended, at least in part, in favor of better frame rate and/or resolution (e.g., image quality) in B-mode scanning to assist in the search. In response to the transducer being used to scan the located region, the mixed mode imaging is performed and the B-mode quality and/or frame rate may decrease.

The switching between enabling the mixed mode for scanning and suspending at least part of the mixed mode for searching occurs automatically. Rather than relying on user touches of the control to switch between modes, the system assists the searching despite being configured for mixed mode imaging and then automatically returns to mixed mode imaging once the search is complete. The determination of transducer use and corresponding imaging mode may occur without user entry of mode selection after configuring for mixed mode operation.

The method is implemented by the system of FIG. 3 or a different system. For example, the user configures the ultrasound system for mixed mode operation. A processor, controller, or image processor of an ultrasound imaging system determines the mode of use of the transducer, automatically switches modes, and adapts filtering. The ultrasound scanner performs the appropriate imaging. Beamformers, memory, detectors, and/or other devices may be used to acquire the data, perform one or more of the acts, and/or output the data. The processor may control the devices to perform the method of FIG. 1.

Additional, different, or fewer acts may be provided. For example, the method is performed without adaptive filtering in act 40.

The acts are performed in the order described or shown, but may be performed in other orders. The adaptive filtering of act 40 is performed as part of or simultaneously with the imaging of acts 32 and 36, but may be performed at other times. The detection of act 28 occurs during either of the imaging of acts 32 or 36, but may be performed separately or in sequence with the imaging (e.g., interleaving detection and imaging). Once the mode of use of the transducer is determined and imaging mode switched, then the selected imaging is performed. The mode of use detection may be ongoing or periodic during the imaging in a given mode. Upon a transition in the mode of use, the switch is performed again and different imaging is then performed. The mode of use detection may be ongoing or periodic during the selected imaging mode.

In act 26, the user configures the ultrasound system for mixed mode imaging. The user depresses a key, selects a menu item, or otherwise inputs an indication that imaging in a mixed mode is desired. The user may make a combination of selections to configured for mixed mode, such as activating B-mode, defining a field of view, indicating a region of interest in the field of view, and activating color flow for the region of interest. Alternatively, the ultrasound system boots up or configures itself for mixed mode imaging based on presets or other user selections.

Any mixed mode may be used. The mixed mode is a combination of two or more ultrasound imaging modes. For example, the mixed mode is a combination of B-mode imaging and color flow (e.g., Doppler) imaging. B-mode is used for tissue response and color flow is used for fluid response. The color flow information may be for a region of interest or sub-set of the B-mode field of view. Alternatively, the same fields of view are used.

Other modes include contrast agent, harmonic, pulsed wave Doppler (i.e., spectral Doppler), M-mode, elastography, other parametric imaging mode, Doppler tissue (i.e., tissue motion), or other ultrasound imaging mode. The different modes detect different types of information. For mixed mode, any two or more modes are performed together, such as simultaneously or in an interleaved manner. Image information from the two or more modes is presented in a same image or adjacent images.

The user may input gate, region of interest, and/or field of view settings for one or more of the modes in the mixed mode configuration. Other settings appropriate for a given mode may be input, such as selection of velocity or power for color flow, frequency for B-mode, or any other mode specific setting. Alternatively, defaults or processor-determined settings are used.

The configuration is for the data path in the ultrasound system. The configuration may include control settings for the transmit beamformer, receive beamformer, filter, detector, image processor, scan converter, display map, other device, or combinations thereof. The scanning and imaging of the ultrasound system is configured for operating in the mixed mode.

Once configured for mixed mode imaging, the sonographer begins or continues scanning the patient. The overall process for scanning and imaging is placing the transducer on the patient, rotating and/or translating the transducer to locate a region within the patient, and then recording an image or images of the region. Images from other angles and/or of other regions may be desired for diagnosis or therapy, so the transducer is again rotated and/or translated to locate a desired position for recording images with the transducer held stationary. In this process, the transducer may or may not be lifted from the patient. For difficult to image patients, the sonographer may use both hands to apply sufficient pressure during the scanning of the region of interest and/or during the searching for the region of interest.

The transducer is used in two modes, searching (e.g., survey or seek) mode and scan (e.g., record or stationary) mode. Images are generated in both modes, but for different purposes. During searching, a greater frame rate and/or resolution for viewing anatomic detail is desired. During the scan mode, the configured mixed mode is desired.

Rather than the user reconfiguring the system through the user interface at a time of occurrence of each of these different search and scan modes of transducer use, the differences in imaging mode may be provided automatically while configured as desired for the scan mode (i.e., while configured in the mixed imaging mode).

In act 28, the mode of use of the transducer is detected. A processor distinguishes between a search for a region of interest (i.e., search mode of use) and imaging the region of interest (i.e., scan mode of use). User input of the mode of use using a button or selection is avoided. Instead, the processor detects position and/or motion of the transducer to automatically detect the mode of use. For example, the processor distinguishes based on input from a transducer motion sensor, ultrasound data indicating motion, or both.

In one embodiment, motion of the transducer, of the patient being scanned by the ultrasound transducer, or both is used to detect the mode of transducer use. Due to patient and/or transducer shift, the field of view also shifts. The plane or volume being imaged within the patient changes position within the patient because of the motion. For example, the sonographer may be searching for the region of interest, so moves the transducer around on the skin surface of the patient. Resulting images are viewed during the movement until the region (e.g., part of an organ) is located. Then, the amount of movement is reduced or movement ceases to generate images of the organ at the located region. The scan mode may correspond to stationary or substantially stationary positioning of the transducer. “Substantially” is used to account for unintended motion.

Another source of motion is movement within the patient. For example, heart and/or lung movement causes organs or other tissue to shift position relative to other organs and/or other tissue. Some internal tissues of the patient may move, such as the heart or heart walls moving cyclically. Greater temporal resolution (e.g., higher frame rate) is desired during transducer or tissue motion. Tissue movement may indicate a search mode.

For on-going imaging, the motion determination is repeated continuously or periodically, such a repeating for each or other integer number of images in a sequence. Alternatively, the motion is determined in response to user activation or other trigger.

The amount of motion is determined. The motion vector in one, two, or three-dimensions is calculated by the processor. The translation or lateral shift is found. In other embodiments, the amount of rotation and/or change in scale is determined. The magnitude of the motion indicates how much motion or change in position has occurred. Any period for determining the motion may be used, such as motion between sequential frames or images or motion between the same phases of different cycles in the heart cycle.

The motion is determined from any source. A sensor is used in one embodiment. For example, a magnetic position sensor on the transducer is used to detect motion of the transducer. In other embodiments, ultrasound data is used to detect the motion. Combinations of techniques for detecting motion may be used.

For detecting the motion from ultrasound data, B-mode data may be used. Two frames or images of B-mode data are relatively displaced by different amounts by the processor. For each displacement, a correlation of some (sparse sampling or region of interest) or all of the data is calculated. The displacement (e.g., translation with or without rotation) with the greatest correlation provides the motion vector. In another embodiment, de-correlation indicates motion in an elevation direction. An amount of de-correlation between sequential frames or images without relative offset shows an amount of out-of-scan plane motion. In yet another embodiment, Doppler data is used. A global velocity is calculated, such as an average tissue velocity in an image or part of an image. The global velocity represents the amount of motion.

In one approach, the ultrasound data also used for mixed mode imaging is used to detect the motion. The mixed mode imaging acquires data, such as B-mode or color flow data. This data is used for correlation or de-correlation. The B-mode data from B-mode scanning with part or all of the mixed mode suspended may be used to detect motion (i.e., during the search mode). B-mode data acquired for motion detection and not for imaging may be used. Other types of data acquired with the mixed mode suspended in part or total may be used to detect the motion.

Motion as measured between any two times may be used. Alternatively, the motion is detected multiple times over time. For example, global displacement over time is detected using two or more detected motions (i.e., data from three or more times). A profile of the amount of global displacement over time is obtained. Any period may be used for the profile. The velocity, average, or other measure of motion (e.g., maximum of the profile) is calculated from the profile to provide the amount of motion. The magnitude of motion is determined from the profile.

A threshold is applied in the detection of the motion. If the motion is over the threshold, then the transducer is in search mode. If the motion is below the threshold, then the transducer is in scan mode. For motion equal to the threshold, either of the modes may be indicated. Any threshold may be used, such as a preset, predetermined, or other threshold.

Other information may additionally or alternatively be used to distinguish the mode of use of the transducer. For example, contact of the transducer with tissue and/or brightness from the ultrasound data is used. Contact of the transducer may be measured with an electric field, capacitive sensing, or contact sensor. Alternatively, contact is measured based on ultrasound data, such as calculating an average near field intensity. Low intensity indicates scanning air, not tissue. Brightness likewise indicates scanning air rather than tissue. The level of contact may be determined, such as sensing contact for each of different near field regions across the face of the transducer. The transducer being partially separated from the tissue may be detected.

The transducer not in contact with the tissue indicates a search mode or use other than scanning. Once contact is established, the search mode may be assumed for a given period, such as for 10 seconds. Loss of contact is also treated as entering the search mode. The contact or brightness may be used in combination with the motion detection, such as triggering search mode based on either loss of contact or motion above a threshold and triggering scan mode based on motion below a threshold after re-establishing contact.

Where the detected mode of use of the transducer is the same as the most recent detected mode, the current imaging continues. When the detected mode of use changes, then the imaging changes. For example, the motion of the transducer transitions from below to above or from above to below the threshold. This transition indicates a change in the mode of use of the transducer from a scan mode of use to a search mode of use, or vice versa. A corresponding change is made for the imaging mode.

In act 30, where a transition occurs, the ultrasound scanner automatically switches to scanning the patient in the appropriate mode, such as switching from B-mode to mixed mode or from mixed mode to B-mode. For example, in response to the detecting that the motion is now below the threshold, the processor causes the ultrasound scanner to operate in the mixed mode instead of the B-mode or other partial mode of the mixed mode. Just B-mode scanning or just another mode of scanning is used for searching, then the system automatically transitions to the mixed mode with B-mode and another mode of imaging or with a combination of other modes of imaging not including B-mode.

The transmit beamformation, receive beamformation, detection, filtering, scan conversion, and/or other process changes to transition. For example, B-mode imaging uses a sequence of transmit beams and corresponding receive beams to scan a region at a given frequency. In a mixed B-mode and color flow mode, multiple transmissions and receptions along each scan are used in a flow sample count to estimate the velocity, variance, and/or power at a given location. The transmit and receive beamformation sequence, waveforms, scan angle, depth range, and/or other characteristic changes to interleave the color flow scanning with the B-mode scanning. Similarly, the Doppler or flow estimator is not used for B-mode imaging, but is used for color flow imaging.

Where one mode of imaging is common to both the mixed mode and the imaging used in searching (e.g., B-mode used for searching and for combined B-mode and color flow mode), the settings for the B-mode imaging stay the same or change to account for interleaving with another mode or modes in the mixed mode. The transition is implemented by changing a value or values for acquisition or other parameters. The frame rate may be different. For a different frame rate, the pulse repetition interval, scan line density, depth, scan angle for the field of view, amount of filtering, or other process is varied. The spatial resolution may be different. For different spatial resolution, the scan line density, sample density along scan lines, scan angle for the field of view, or other characteristic is varied. Both temporal and spatial resolution may be varied.

The ultrasound scanner may have limited processing resources or the speed of sound in tissue may be a limiting factor. As a result, just B-mode imaging for searching may have greater spatial and/or temporal resolution due to not sharing resources or time with another mode. Only the B-mode part of the mixed mode is used for searching while one or other modes of the mixed mode are suspended. For mixed mode imaging, the spatial and/or temporal resolution for the B-mode part of the mixed mode imaging may be reduced due to sharing the resources with another mode. In alternative embodiments, B-mode imaging is not provided as part of the mixed mode.

The switch occurs without operator input into a user interface of an ultrasound system while performing the scanning. The operator may configure the ultrasound system for mixed mode scanning in act 26 using the user interface, but further user input other than moving the transducer is not used to switch between mixed mode imaging for the scan mode of use of the transducer and B-mode or other mode of imaging for the search mode of use of the transducer. Rather than the user reconfiguring the system on the fly, the system automatically transitions between the mixed mode and B-mode (or other search mode) without the user having to depress a button, alter a knob, slide a slider, or other touching of the user interface. After configuration in the mixed mode, the switch is automatic or without user interaction with the user interface while scanning the patient.

In act 32, the ultrasound scanner performs B-mode imaging for searching. When the mode of use of the transducer is detected as searching, then B-mode imaging is performed. In one embodiment, only B-mode or only one mode of imaging is provided for searching. By scanning just in B-mode or one mode, greater processing, memory, or other resource of the ultrasound scanner is available for that mode, allowing greater spatial and/or temporal resolution or field of view than if the imaging included other modes. In yet other alternative embodiments, a different mode than B-mode is used for searching, such as searching flow with color flow imaging while one or more other modes (e.g., spectral Doppler or B-mode) of the mixed mode imaging are suspended or not performed.

The ultrasound scanner is configured to scan in the mixed mode, yet one or more of the modes are not performed since the search overrides the configuration. The ultrasound scanner scans only in B-mode or other search mode. Any other modes in the mixed mode are not performed during the searching. Where the mixed mode includes B-mode and one or more other modes, the one or more other modes are suspended or not performed during the searching despite the scanner being configured for mixed mode scanning. In alternative embodiments, one or more of the other modes continue, but at a lesser spatial and/or temporal resolution to allow greater B-mode spatial and/or temporal resolution than would be provided with full or as configured mixed mode imaging.

For B-mode or other mode of imaging, the ultrasound scanner scans as appropriate for that mode. The scan format, sequence, waveforms, or other beamformer settings provide the scanning. The data processing of the scan data is as appropriate for that mode. For example, beamformed samples for locations in the patient are detected, such as using intensity or B-mode detection. Spatial and/or temporal filtering may or may not be applied. The detected B-mode data is scan converted and mapped to grey scale display values.

In act 34, one or more B-mode or other images are generated for searching. Based on the scanning and image processing for B-mode or other mode, an image is generated. The image includes red, green, blue (RGB) or other display values formatted for the display (e.g., Cartesian coordinate). For viewer perceived gray scale imaging, the RGB values may be approximately equal. Graphics may or may not be provided as part of the image, such as indicating settings (e.g., frequency) used to scan.

The image represents the region of the patient in the B-mode or other search mode field of view. The image is a one, two, or three-dimensional representation. For example, data from a scan of a volume is rendered using surface or volume rendering to a two-dimensional display. As another example, data representing a scan plane is used to generate an image of the scan plane.

The frame rate and/or resolution of the imaging are based on the scan and/or image processing settings. The frame rate and/or resolution of the generated images are the same or different for search as for scanning. For example, B-mode images are generated in both of acts 34 and 38. The B-mode images generated in act 34 have a greater spatial and/or temporal resolution and/or field of view than the B-mode images generated in act 38.

The imaging of act 32 and generation of images in act 34 are on going or repeated during a same imaging session (e.g., for a same patient in a same 15 minute ultrasound examination). The imaging continues to allow the sonographer to search for a region of the patient to be scanned for diagnosis or treatment. A sequence of any number of images is generated.

When the mode of use transitions to the scan usage as detected in act 28, the imaging is switched in act 30 to imaging in the mixed mode in act 36. Once motion or other indicator that the location of the region to be used for diagnosis or therapy is found, the mode of use of the transducer switches from search to scan.

In act 36, the ultrasound scanner performs mixed mode imaging for scanning or recording. When the mode of use of the transducer is detected as scanning, then mixed mode imaging is performed. Two or more modes of imaging are performed simultaneously or in an interleaved manner. For interleaving, scan line-by-scan line, groups of scan lines, frame-by-frame, or groups of frame interleaving is used. For example, a frame of B-mode information is acquired interleaved with acquiring data for a plurality of frames of color flow data.

By scanning in mixed mode, lesser processing, memory, or other resource of the ultrasound scanner is available for a given mode. The processing, memory, or other resources are distributed between the different modes of the mixed mode as configured in act 26. The ultrasound scanner is configured to scan in the mixed mode. That configuration is used once the region to be scanned is located. The searching no longer overrides the mixed mode configuration. The ultrasound scanner scans for two or more modes.

For each mode of imaging, the ultrasound scanner scans as appropriate for that mode. The scan format, sequence, waveforms, or other beamformer settings provide the scanning. The data processing of the scan data is as appropriate for that mode. For example, beamformed samples for locations in the patient are detected, such as using intensity or B-mode detection. Spatial and/or temporal filtering may or may not be applied. The detected B-mode data is scan converted and mapped to grey scale display values. As another example, the scanning provides samples for a flow sample count at a flow sample interval for estimating the velocity, variance, and/or energy of flow or tissue motion. A corner turning memory operates in conjunction with the Doppler processor to isolate fluid or tissue motion information for estimation. The estimated flow is scan converted and mapped to colors for display.

In act 38, one or more mixed mode images are generated. Based on the scanning and image processing for each mode, an image is generated. The image includes red, green, blue (RGB) or other display values formatted for the display (e.g., Cartesian coordinate). Graphics may or may not be provided as part of the image, such as indicating settings (e.g., frequency) used to scan.

The image represents the region of the patient in two or more modes. The different modes represent the same or different locations in the region. For example, locations associated with flow (at least within a region of interest) are represented by color flow information and locations associated with tissue are represented by gray scale B-mode information. The image represents a field of view. The information from the different modes overlaps in the sense of representing the same region of the patient. The different modes may result in non-overlapping display. For example, M-mode, color M-mode, or pulse wave Doppler provide strip displays for a gate or line. The strip display is adjacent to but not overlapping with a B-mode and/or color flow image. Both representations are created on a same display or part of a same image. The information from both modes is displayed at a same time to the user. In alternative embodiments, the images from the different modes are displayed separately and/or sequentially.

The image is a one, two, or three-dimensional representation. For example, data from a scan of a volume is rendered using surface or volume rendering to a two-dimensional display. As another example, data representing a scan plane is used to generate an image of the scan plane.

The frame rate and/or resolution of the imaging are based on the scan and/or image processing settings. The frame rate and/or resolution of the generated images are the same or different for mixed mode as for the mode limited searching. For example, B-mode images are generated in both of acts 34 and 38. The B-mode images generated in act 34 have a greater spatial and/or temporal resolution than the B-mode images generated in act 38. Due to the mixed mode imaging, fewer resources may be available for B-mode scanning, so the B-mode images have a lesser temporal or spatial resolution in act 38 than for act 34.

The imaging of act 36 and generation of images in act 38 are on-going or repeated during a same imaging session (e.g., for a same patient in a same 15 minute ultrasound examination). The imaging continues to allow the sonographer to acquire the desired information for diagnosis or therapy. A sequence of any number of images is generated.

When the mode of use of the transducer transitions to the search again as detected in act 28, the imaging is switched in act 30 to search imaging in act 32. Once motion or other indicator that searching has begun again occurs (e.g., motion transitions to be above the threshold), the mode of imaging automatically switches from scan to search. As long as the motion is below the threshold or other transducer use sensing indicates scanning for the mixed mode, then the mixed mode imaging continues.

In act 40, the filtering adapts to the mode of use of the transducer. For example, the filtering adapts as a function of the motion. The adaptation may be binary, such as filtering at one level for searching and filtering at a different level for scanning. In other embodiments, the adaptation has more levels than switching between the imaging for searching and the imaging for mixed mode scanning. For example, the amount of filtering is linearly or non-linearly mapped to three or more levels based on corresponding different amounts of motion. Similarly, different combinations of modes of imaging may be provided for different amounts of motion, providing more than two options for automated transition.

The filtering is for the same mode of imaging, but varies based on the mode of use of the transducer. For example, B-mode imaging for searching is filtered differently than B-mode imaging used in mixed mode imaging.

The adaptation is in amount and/or type of temporal and/or spatial filtering. For example, less temporal and spatial filtering are provided for B-mode imaging while searching than for B-mode imaging in mixed mode. For mixed mode, the color flow or other mode may be more important while B-mode is provided for tissue reference information. Accordingly, more temporal and/or spatial filtering may be provided to remove noise. Conversely, searching results in more rapidly changing views. The increased spatial and/or temporal resolution is important for searching, so less temporal and/or spatial filtering is provided.

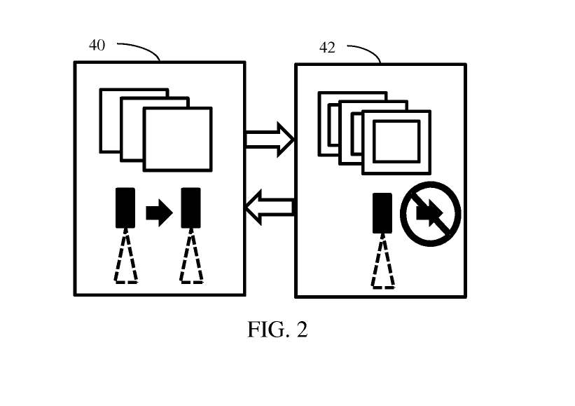

FIG. 2 illustrates one example of the method of FIG. 1. At box 40, a sequence of images is acquired while moving the transducer to scan different locations. At box 42, a sequence of images is acquired with the transducer held substantially stationary. “Substantially” is used to account for unintended sonographer or patient motion. The mixed mode images include a color flow region of interest with B-mode imaging, but other mixed mode images may be used. The search images of box 40 are just B-mode images, but may be obtained from another mode. Based on detected transducer use and/or motion, either of box 40 or 42 is provided at any given time. The imaging may transition from box 40 to box 42 or vice versa.

FIG. 3 shows one embodiment of a system 10 for acquisition control for mixed mode ultrasound imaging. The ultrasound system is configured for mixed mode imaging. Rather than maintain that mixed mode imaging but without requiring user-based reconfiguration, the system 10 automatically switches between one combination of modes different from the configured mixed mode and the mixed mode. The switch occurs in response to a data or transducer-based indication of the user searching to locate a plane of interest for diagnosis. During the search, a single mode or different combination of modes is used. Once the search is complete, the ultrasound system automatically transitions back to the configured mixed mode imaging.

The system 10 is a medical diagnostic ultrasound imaging system. In alternative embodiments, the system 10 is a personal computer, workstation, PACS station, or other arrangement at a same location or distributed over a network for real-time or post acquisition imaging through connection with a beamformer and transducer.

The system 10 implements the method of FIG. 1, the approach of FIG. 2, or other methods. The system 10 includes a transmit beamformer 12, a transducer 14, a receive beamformer 16, an image processor 18, a display 20, a memory 22, and a processor 24. Additional, different or fewer components may be provided. For example, a user input is provided for manual or assisted designation of a region of interest within a field of view for mixed mode imaging and/or for configuring the ultrasound system 10 for mixed mode imaging.

The transmit beamformer 12 is an ultrasound transmitter, memory, pulser, analog circuit, digital circuit, or combinations thereof. The transmit beamformer 12 is configured to generate waveforms for a plurality of channels with different or relative amplitudes, delays, and/or phasing. The waveforms are generated and applied to a transducer array with any timing or pulse repetition frequency. For example, the transmit beamformer 12 generates a sequence of pulses for B-mode scanning in a linear, sector, or Vector® format. As another example, the transmit beamformer 12 generates a sequence of pulses for color flow scanning, such as pulses for forming 2-12 beams in an ongoing flow sample count per scan line for a region of interest within a B-mode field of view.

The transmit beamformer 12 connects with the transducer 14, such as through a transmit/receive switch. Upon transmission of acoustic waves from the transducer 14 in response to the generated waves, one or more beams are formed during a given transmit event. The beams are for B-mode, color flow mode, and/or other modes of imaging. A sequence of transmit beams are generated to scan a one, two or three-dimensional region. Sector, Vector®, linear, or other scan formats may be used. The same region is scanned multiple times.

The transducer 14 is a 1-, 1.25-, 1.5-, 1.75- or 2-dimensional array of piezoelectric or capacitive membrane elements. The transducer 14 includes a plurality of elements for transducing between acoustic and electrical energies. For example, the transducer 14 is a one-dimensional PZT array with about 64-256 elements.

The transducer 14 connects with the transmit beamformer 12 for converting electrical waveforms into acoustic waveforms, and connects with the receive beamformer 16 for converting acoustic echoes into electrical signals. The transducer 14 transmits beams. To form the beams, the waveforms are focused at a tissue region or location of interest in the patient. The acoustic waveforms are generated in response to applying the electrical waveforms to the transducer elements. For scanning with ultrasound, the transducer 14 transmits acoustic energy and receives echoes. The receive signals are generated in response to ultrasound energy (echoes) impinging on the elements of the transducer 14.

The receive beamformer 16 includes a plurality of channels with amplifiers, delays, and/or phase rotators, and one or more summers. Each channel connects with one or more transducer elements. The receive beamformer 16 applies relative delays, phases, and/or apodization to form one or more receive beams in response to each transmission for detection. Dynamic focusing on receive may be provided. Relative delays and/or phasing and summation of signals from different elements provide beamformation. The receive beamformer 16 outputs data representing spatial locations using the received acoustic signals. In alternative embodiments, the receive beamformer 16 is a processor for generating samples using Fourier or other transforms.

The receive beamformer 16 may include a filter, such as a filter for isolating information at a second harmonic, transmit (i.e., fundamental), or other frequency band relative to the transmit frequency band. Such information may more likely include desired tissue, contrast agent, and/or flow information. In another embodiment, the receive beamformer 16 includes a memory or buffer and a filter or adder. Two or more receive beams are combined to isolate information at a desired frequency band, such as a second harmonic, cubic fundamental, or other band.

The receive beamformer 16 outputs beam summed data representing spatial locations. Data for a single location, locations along a line, locations for an area, or locations for a volume are output. The data may be for different purposes. For example, different scans are performed for B-mode or tissue detection than for color flow mode detection. Alternatively, the B-mode data is also used to for color flow detection.

The image processor 18 is a B-mode detector, Doppler detector, pulsed wave Doppler detector, correlation processor, Fourier transform processor, filter, other now known or later developed processor for implementing an imaging mode, or combinations thereof. The image processor 18 provides detection for the imaging modes, such as including a Doppler detector (e.g., estimator) and a B-mode detector. A spatial filter, temporal filter, and/or scan converter may be included in or implemented by the image processor 18. The image processor 18 outputs display values, such as detecting, mapping the detected values to display values, and formatting the display values or detected values into a display format. The image processor receives beamformed information and outputs image data for display.

The processor 24 is a control processor, general processor, digital signal processor, graphics processing unit, application specific integrated circuit, field programmable gate array, network, server, group of processors, data path, combinations thereof, or other now known or later developed device for detecting transducer use and controlling the ultrasound system 10 to image accordingly. The processor 24 is separate from or part of the image processor 18. As a separate device, the processor 24 requests, receives, accesses, or loads data at any stage of processing (e.g., beamformed, detected, scan converted, display mapped or other stage) for detecting and controlling. The processor 24 is configured by software and/or hardware to perform or cause performance of the detecting, switching, imaging, and/or image generation acts.

The processor 24 is configured to cause the transmit and receive beamformers 12, 16 to operate in the B-mode and not the color flow mode during transducer motion or other searching use and to operate in both the B-mode and color flow mode for stationary positioning or other diagnostic or therapy scan use of the transducer 14. Other searching modes than B-mode may be used. Other mixed modes than B-mode and color flow mode may be used.

The processor 24 or a separate beamformer controller configures the beamformers 12, 16. By loading values into registers or a table used for operation, the values of acquisition parameters used by the beamformers 12, 16 for imaging are set. Any control structure or format may be used to establish the imaging sequence. The beamformers 12, 16 are caused to acquire data for imaging at a frame rate and/or with a resolution. Different values of one or more acquisition parameters may result in a different frame rate and/or resolution.

The processor 24 is configured to determine the use of the transducer 14. Correlation of ultrasound data from different times, de-correlation of the ultrasound data, tissue contact by the transducer 14, brightness measures of the image, or other information is used. Sensors may connect with the processor 24, and/or ultrasound data is used to determine the use.

The processor 24 is configured to switch the ultrasound imaging system 10 between the search mode (e.g., B-mode) and the mixed mode based on the use of the transducer 14. The switch is automatic. The user configures the ultrasound system 10 for mixed mode operation. The mixed mode is the desired mode for diagnosis and/or therapy. The ultrasound system 10, under the control of the processor 24, suspends the mixed mode, despite the configuration, for searching and then implements the mixed mode upon locating the region to be diagnosed or treated. The switch occurs without user input to switch between the operations for searching and for mixed mode imaging. The user does not directly activate the change in modes, but instead activates the desired mixed mode, and the processor 24 later switches.

The processor 24 or image processor 18 generates and outputs image or display values to the display 20. For example, B-mode images or mixed mode images are output. Text, numerical indication, or graphic may be added and displayed to the user. A graph may be displayed.

The display 20 is a CRT, LCD, monitor, plasma, projector, printer, or other device for displaying an image or sequence of images. Any now known or later developed display 20 may be used. The display 20 is operable to display one image or a sequence of images. The display 20 displays two-dimensional images or three-dimensional representations.

During searching, the display 20 displays images from a single mode or mixed mode different than the mixed mode for which the system 10 is configured, such as displaying one or more B-mode images without a color flow image during the transducer motion. During scanning after locating the sought region of the patient, the display 20 displays mixed mode images, such as displaying combination B-mode and color flow images for the stationary positioning of the transducer 14.

The spatial resolution, image quality, and/or frame rate are based, in part, on the acquisition and/or imaging processing parameters. Where a mode is common to both the searching and the mixed mode, the spatial resolution, image quality, and/or frame rate may be different during searching than during mixed mode imaging. For example, the B-mode images during transducer motion having greater frame rate and/or spatial resolution than the B-mode for the stationary positioning of the transducer 14.

The image processor 18, processor 24, the receive beamformer 16, and the transmit beamformer 12 operate pursuant to instructions stored in the memory 22 or another memory. The instructions configure the system for performance of the acts of FIG. 1. The instructions configure the image processor 18, the processor 24, the receive beamformer 16, and/or the transmit beamformer 12 for operation by being loaded into a controller, by causing loading of a table of values (e.g., elasticity imaging sequence), and/or by being executed.

The memory 22 is a non-transitory computer readable storage media. The instructions for implementing the processes, methods and/or techniques discussed herein are provided on the computer-readable storage media or memories, such as a cache, buffer, RAM, removable media, hard drive or other computer readable storage media. Computer readable storage media include various types of volatile and nonvolatile storage media. The functions, acts, or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing, and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for acquisition control for mixed mode ultrasound imaging, the method comprising:
scanning a patient in a B-mode with an ultrasound transducer;
generating B-mode images from the scanning in the B-mode;
detecting that motion of the ultrasound transducer, the patient being scanned by the ultrasound transducer or both has transitioned to below a threshold;
automatically switching to scanning the patient in a mixed mode in response to the detecting that the motion is below the threshold, the mixed mode different from the B-mode; and
generating mixed mode images from the scanning in the mixed mode.

2. The method of claim 1 wherein detecting the motion comprises correlating between frames of B-mode data from the scanning in the B-mode.

3. The method of claim 1 further comprising detecting that the motion has transitioned to above the threshold and automatically switching to the scanning of the patient in the B-mode and not the mixed-mode.

4. The method of claim 1 wherein detecting the motion comprises detecting as a function of tissue contact, brightness, or combinations thereof.

5. The method of claim 1 wherein scanning in the B-mode comprises scanning in only B-mode.

6. The method of claim 1 wherein scanning in the mixed mode comprises scanning with a combination of two or more modes.

7. The method of claim 1 wherein scanning in the mixed mode comprises scanning in the B-mode interleaved with scanning in a color flow, M-mode, and/or pulse wave Doppler mode.

8. The method of claim 7 wherein scanning in the B-mode interleaved with scanning in the color flow, M-mode, and/or pulse wave Doppler mode comprises scanning with a lesser temporal and/or spatial resolution than scanning in the B-mode not part of the mixed mode.

9. The method of claim 1 wherein automatically switching comprises switching without operator input into a user interface of an ultrasound system performing the scannings.

10. The method of claim 1 wherein detecting and automatically switching comprises distinguishing between a survey to find a region of interest using just B-mode and scanning at the region of interest in the mixed mode.

11. The method of claim 1 further comprising adapting filtering as a function of the motion.

12. A non-transitory computer readable storage medium having stored therein data representing instructions executable by a programmed processor for acquisition control for mixed mode ultrasound imaging, the storage medium comprising instructions for:
distinguishing between a search for a region of interest and imaging the region of interest, the distinguishing being based on input from a transducer motion sensor, ultrasound data, or both;
imaging in only B-mode for the search; and
imaging in the mixed mode for the imaging of the region of interest.

13. The non-transitory computer readable storage medium of claim 12 wherein distinguishing comprise distinguishing based on correlation of ultrasound data acquired at different times.

14. The non-transitory computer readable storage medium of claim 12 wherein distinguishing comprises distinguishing based on an amount of motion, contact of a transducer with tissue, or brightness from the ultrasound data.

15. The non-transitory computer readable storage medium of claim 14 wherein imaging in only the B-mode comprises B-mode imaging for the search and switching to the imaging in the mixed mode once motion indicates location of the region of interest.

16. The non-transitory computer readable storage medium of claim 12 wherein imaging in the mixed mode comprises imaging with the B-mode in combination with another mode.

17. The non-transitory computer readable storage medium of claim 16 wherein imaging for the B-mode in the mixed mode has a lesser spatial and/or temporal resolution than imaging for only the B-mode.

18. The non-transitory computer readable storage medium of claim 12 further comprising adapting filtering as a function of the search for and the imaging of the region of interest.

19. A system for acquisition control for mixed ultrasound imaging, the system comprising:
a transmit beamformer and a receive beamformer configured to scan, with a transducer, a patient with ultrasound in B-mode and color flow mode;
a processor configured to cause the transmit and receive beamformers to operate in the B-mode and not the color flow mode during transducer motion and to operate in both the B-mode and color flow mode for stationary positioning of the transducer; and
a display operable to display a B-mode image without a color flow image during the transducer motion and to display both the B-mode image and the color flow image for the stationary positioning of the transducer.

20. The system of claim 19 wherein the processor is configured to cause the transmit and receive beamformers to operate in the B-mode and not the color flow mode and in both the B-mode and color flow modes without user input to switch between the operations, the B-mode during the transducer motion having a greater frame rate and/or spatial resolution than the B-mode for the stationary positioning of the transducer.
